(19) United States
(12) Patent Application Publication
(10) Pub. No.: US 2011/0129871 A1
(43) Pub. Date: Jun. 2, 2011

(54) CULTURE MEDIUM ENABLING STAPHYLOCOCCUS AUREUS TO BE DIFFERENTIATED FROM COAGULASE-NEGATIVE STAPHYLOCOCCI

(75) Inventors: David Mosticone, Sainte-Consorce (FR); Sylvain Orenga, Neuville Sur Ain (FR); Antoine Vimont, Lyon (FR); Yuping Guo, Tassin la Demi Lune (FR); Martine Dhedin, Sain Bel (FR)

(73) Assignee: BIOMERIEUX, Marcy L'Etoile (FR)

(21) Appl. No.: 13/056,208

(22) PCT Filed: Aug. 13, 2009
(86) PCT No.: PCT/FR2009/051588

§ 371 Date: Jan. 27, 2011

(30) Foreign Application Priority Data
Aug. 13, 2008 (FR) 0855555

(51) Int. Cl. C12Q 1/14; C07F 9/117; C07F 9/655; C07F 9/572; C12N 1/20; C12Q 1/02

(52) U.S. Cl. 435/36; 568/15; 549/220; 548/414; 435/252.1; 435/29

(57) ABSTRACT

A specific culture medium for growth, detection, identification and/or counting of Staphylococcus aureus bacteria, said medium includes at least one fluorogenic, chromogenic or luminescent substrate of phospholipase C. Said medium permits differentiation between Staphylococcus aureus and coagulase-negative staphylococci.

The present invention relates in general to the field of microbiological analysis. More particularly, the present invention relates to a selective culture medium for growth, detection, identification and/or counting of staphylococci, allowing Staphylococcus aureus to be differentiated from coagulase-negative staphylococci.

The bacteria of the genus Staphylococcus or staphylococci are responsible for a large number of nosocomial infections and represent a considerable problem in hospitals. These bacteria are Gram-positive cocci, which can be classified in two main groups that are distinguished by the production of a protein, coagulase, which triggers the coagulation of plasma. Thus, a distinction is made between coagulase-negative staphylococci, the main representative of which is Staphylococcus epidermidis, and coagulase-positive staphylococci, the main representative of which is Staphylococcus aureus, which is well known for its virulence. The staphylococci are of wide occurrence in the environment, the skin and mucosae of humans and animals. Regular desquamation from these hosts has the effect of dispersing them widely in nature (water, soil, air, foodstuffs, objects), and in the hospital environment draconian measures of hygiene and isolation of patients are required to limit the spread of the epidermal strains. Thus, Staphylococcus aureus , which represents 80 to 90% of the staphylococci isolated in the clinical setting is a major human pathogen responsible for numerous infections in the hospital environment, such as notably nosocomial pneumonias, infections of surgical wounds, infections of burns, infections of foreign bodies (heart valves, hip prostheses, clamps etc.) and systemic infections or septicaemias which are often due to the use of intravascular catheters, or to dissemination of the bacterium from another site of infection.

The staphylococci, and in particular Staphylococcus aureus , also pose a considerable risk in the area of food. In fact, certain strains of Staphylococcus aureus are capable of producing enterotoxins, whose ingestion by the consumer leads to poisoning, causing nausea, abdominal pains and especially severe, repeated vomiting, often accompanied by diarrhea.

Staphylococcal food poisoning thus represents one of the primary forms of food poisoning of bacterial origin.

Dissemination is generally by animals and humans, whether they are sick or are healthy carriers, by raw milk (mastitis), by the air and contaminated surfaces or equipment in contact with foods and healthy or infected carriers.

The detection and identification of staphylococci and in particular of Staphylococcus aureus in foods therefore constitute a major public health challenge.

Among the culture media used for the detection, identification and counting of bacteria of the genus Staphylococcus, a distinction is made between non-selective media such as Columbia medium or trypticase-soya agar and selective media, such as Chapman agar and Baird-Parker agar. Staphylococcus aureus can also be detected on a blood agar depending on its morphological characteristics and its haemolysis profile but this method is not very sensitive and specific and is used little, if at all, in the food industry. Heart-brain broth is also regularly used for investigation of staphylococci.

Specific bacteriological culture media promote the growth of certain microorganisms and limit that of others. They contain at least one inhibitor of microorganisms other than the target pathogen. The effect of the inhibitors must remain limited on the microorganism of interest, since said microorganism of interest can be damaged in the food preparations that are produced. Chapman agar corresponds to a hypersaline medium on an ordinary nutrient base, using mannitol as substrate, fermentation of which is detected by a pH indicator. Said pH indicator can be a coloured indicator or a fluorescent indicator. The Baird-Parker medium corresponds to a rich nutrient base with added potassium tellurite and lithium chloride, selective agents commonly used for growing Staphylococcus aureus. Lithium chloride is an inhibitor of enterococci. Other chemicals can be combined with potassium tellurite and sodium or lithium chloride: ammonium sulphate, sorbic acid, glycine, polymyxin B.

Nevertheless, analysis of food matrices for potentially toxinogenic staphylococci presents special problems, which limit the practical use of methods developed for clinical practice. In fact, the media commonly proposed also permit the growth and, depending on the required phenotypic character, the detection of coagulase-negative staphylococci, which are not regarded as agents of food contamination.

In the case of the Baird-Parker+RPF (rabbit plasma+bovine fibrinogen) medium, which is the reference medium according to standard ISO 6888-1 and 6888-2, a certain number of drawbacks are generally observed.

Firstly, there are difficulties in reading and sensitivity: false negatives may potentially appear owing to the fact that certain strains of Staphylococcus aureus do not develop on this medium or present a very weak and delayed coagulase, suggesting the presence of coagulase-negative staphylococci. Moreover, false-negative results may also be observed because of interference from the matrix: in fact, for counting low levels of contamination with Staphylococcus aureus, minimal dilution of the sample is carried out ( 1/10), which causes difficulty in reading the halo due to the presence of matrix compounds (e.g. samples of milk and/or milk products: the white colour of casein masks the halo revealing coagulase).

The Baird-Parker+RPF medium necessitates combining a source of thrombin and a source of plasminogen. The latter are obtained from the blood of animals, which poses problems of reliability of supply (quality, quantity, etc.).

Moreover, reading of a halo is not possible in broth (liquid medium) and the contrast between the halo and the medium may be reduced. Finally, in the case of confluent colonies, it is difficult to differentiate those that produce the halo from those that do not produce it.

Secondly, there may also be problems of specificity: in the presence of abundant subsidiary flora (for example Bacillus spp), false-positive results can appear with this type of medium. This is the case for example for counting Staphylococcus aureus in certain meat-based products (dry sausage) and milk products (Munster type cheeses) in which Staphylococcus xylosus is used as starter. Thus, black colonies surrounded by a halo appear on the Baird Parker+RPF medium, colonies that are normally characteristic of Staphylococcus aureus on this medium, but which in fact result from contiguous growth of strains of Staphylococcus xylosus (producing black colonies without a halo) and certain strains of Bacillus generating turbidity, which may be identified as a halo on the agar.

Among the alternative methods, such as chromogenic media, owing to the greater similarities between coagulase-positive staphylococci and coagulase-negative staphylococci, there is no medium that enables these two groups to be discriminated very specifically. In fact, the concepts of differentiation (i.e. substrates and/or sugars that are more or less specific, combined with an inhibitor system) employed in media of this type with the aim of differentiating coagulase-positive staphylococci from coagulase-negative staphylococci, are very often complex and do not provide good discrimination, notably for food samples containing a varied and abundant microbial flora.

The absence of specific substrates or sugars for differentiation between coagulase-positive staphylococci and coagulase-negative staphylococci (or even other bacterial species) means using “aggressive” selective/inhibitory systems, which may alter or inhibit the growth or the enzymatic activity in question of Staphylococcus aureus, potentially leading to false-negative results being obtained.

False-negative results in the case of slight contamination may also be obtained with certain media, because of the dilutions required for avoiding matrix interactions: e.g. pink coloration caused by high levels of phosphatase present in milk products, with 3M™ Petrifilm™ Staph Express Count Plates.

Conversely, media based on the degradation of a substrate or fermentation of a sugar of low specificity for Staphylococcus aureus, associated furthermore with an inhibitory system which is not sufficiently selective, thus lead to false-positive results being obtained.

Finally, certain media are based on the use of two substrates in order to ensure optimum differentiation between Staphylococcus aureus and the other staphylococci and bacterial species. Application of such a concept increases the cost of the medium without providing sufficient specificity, notably in the case of samples obtained from foods.

The enzymes of the phospholipase C type are known and are described in the literature as being present in a large number of microorganisms.

Among the phospholipases C, phosphatidylinositol phospholipase C (PIPLC) from Staphylococcus aureus has been purified and characterized (Phosphatidylinositol-Specific Phospholipase C from Staphylococcus aureus, METHODS IN ENZYMOLOGY, 1981-Vol. 71).

This enzyme has also been described as being a possible virulence factor of Staphylococcus aureus (JOURNAL OF CLINICAL MICROBIOLOGY, Nov. 1989, p. 2451-2454-Vol. 27, No. 11 and INFECTION AND IMMUNITY, Dec. 1993, p. 5078-5089-Vol. 61, No. 12). Moreover, studies for evaluating the conditions in vitro permitting maximum production of active PIPLC in Staphylococcus aureus have also been published (Marques et al.; Growth in Acidic Media Increases Production of Phosphatidylinositol-Specific Phospholipase C by Staphylococcus aureus; CURRENT MICROBIOLOGY Vol. 25 (1992), pp.125-128).

Document EP-0 970 239 B1 describes novel chromogenic substrates permitting detection of PIPLC, secreted by various microorganisms. The examples given describe, on the one hand, the preparation of the various substrates mentioned and, on the other hand, demonstrate the optimum conditions for use of the method (e.g. substrate/enzyme concentration, recommended inducers, etc.). However, no culture medium permitting the detection of Staphylococci is described in this document.

Document EP-1 506 309 B1 describes a culture medium for the detection of a microorganism capable of producing a PIPLC, said culture medium containing a combination of at least one fluorogenic compound and at least one chromogenic compound, capable respectively of generating fluorescence and coloration when they are in contact with the PIPLC. The culture medium is described as permitting, without adding inhibitor, the detection of various bacterial species, such as Listeria monocytogenes, Listeria ivanovii, Bacillus cereus, Bacillus thuringiensis, Bacillus mycoides, Bacillus anthracis, Staphylococcus aureus, Legionella pneumophila, species of Clostridium, Helicobacter pylori, species of Candida and species of Aspergillus. However, no culture medium as such, intended for detecting Staphylococcus aureus, is described in this document. The only culture media described relate to the detection of Listeria monocytogenes and Bacillus group cereus. It appears, moreover, that on all the culture media described, the species of staphylococci and in particular Staphylococcus aureus do not grow.

Document EP-0 949 266 131 describes substrates specific for PIPLC as indicator of bacterial activity in particular of the genus Listeria. Said substrate contains at least one compound able to produce colour or fluorescence. The only culture media described relate to the detection of Listeria monocytogenes. It appears, moreover, that on all the culture media described, the species of staphylococci and in particular Staphylococcus aureus do not grow, owing to their inhibition.

Like other phospholipase C, Phosphatidylcholine Phospholipase C (PCPLC) has also been described as being present in various bacteria, including Staphylococcus aureus (J. G. Songer, Trends in Microbiology, Volume 5, Number 4, April 1997, pp. 156-161(6)).

Document EP-1 219 628 describes novel colorimetric substrates for the detection and identification of microorganisms. These substrates are substrates specific for PCPLC. Nevertheless, this document does not describe substrates for the detection and identification of Staphylococcus aureus , nor ad hoc culture medium.

Thus, it appears that, despite the characterization of PIPLC from Staphylococcus aureus more than 25 years ago and identification of a potential role in the virulence of this bacterial species, no culture medium has ever been described that is specific for the detection and/or counting of Staphylococcus aureus, based on the use of at least one substrate specific for PIPLC, chromogenic, fluorogenic or luminescent, and permitting discrimination between this species and the other species of staphylococci, the inhibition of the strains of Staphylococcus aureus on the media described making their use impossible a priori for such an application. Moreover, no document has described the use of a chromogenic, fluorogenic or luminescent substrate specific for PCPLC, in a specific culture medium for detecting or counting Staphylococcus aureus, the use of said substrate permitting discrimination between this species and the other species of staphylococci.

Bearing in mind the problems posed by the state of the art considered above, one of the essential objectives of the present invention is to supply a culture medium that promotes the growth of staphylococci for the purpose of detecting them and/or identifying them and/or counting them, and permitting discrimination of Staphylococcus aureus from the other species of staphylococci.

Another objective of the present invention is to supply a culture medium that limits the production of false-positive results, notably due to coagulase-negative staphylococci.

Another objective of the present invention is to supply a culture medium permitting better coverage of the target bacteria, notably in the case of law levels of contamination, by the use of a reduced inhibitory system,

Another objective of the present invention is to supply a culture medium permitting simple reading and interpretation, through the use of a single specific substrate.

Another objective of the present invention is to supply a culture medium permitting automation of reading.

Finally, a last objective of the present invention is to supply a culture medium making it possible to reduce the time to return the results, owing to reduced selectivity promoting the growth of Staphylococcus aureus.

These objectives, among others, are achieved by the present invention, which relates firstly to a specific culture medium for the growth, detection, identification and/or counting of Staphylococcus aureus bacteria, said medium being characterized in that it comprises at least one chromogenic, fluorogenic, or luminescent substrate of phospholipase C.

“Substrate” means any molecule capable of producing, directly or indirectly, a detectable signal due to enzymatic or metabolic activity of the microorganism.

The substrate can notably be an enzymatic substrate, i.e. a substrate that can be metabolized by an enzyme into a product permitting the direct or indirect detection of a microorganism.

This substrate notably comprises a first moiety specific to the enzymatic activity to be detected and a second moiety serving as marker, called the marker moiety hereinafter. This marker moiety is chromogenic, fluorogenic, luminescent. Preferably, the substrates used in the present invention are fluorogenic. We may mention the derivatives of coumarins and notably umbelliferone, naphthol, resorufin, fluorescein.

In the sense of the present invention, this substrate can be a substrate of phosphatidylinositol phospholipase C (PIPLC). In this case, the concentration of PIPLC substrate in the medium is between 0.01 and 1 g/l.

Preferably, the substrate(s) of PIPLC are taken from the group comprising: 4-nitrophenyl myo-inositol-1-phosphate, 4-methylumbelliferyl myo-inositol-1-phosphate, 3-chloro-7-hydroxy-4-methylcoumarin myo-inositol-1-phosphate, 3-ethoxycarbonyl-4-methylcoumarin myo-inositol-1-phosphate, 3-cyano-4-methylcoumarin myo-inositol-1-phosphate.

According to an alternative, the substrate can be a substrate of phosphatidylcholine phospholipase C (PCPLC). In this case, the concentration of PCPLC substrate in the medium is between 0.01 and 1 g/l.

Preferably, the substrate(s) of PCPLC are taken from the group comprising: 5-bromo-4-chloro-3-indoxyl choline phosphate, 3-indoxyl choline phosphate, 4-methylumbelliferyl choline-phosphate.

In a preferred embodiment, the culture medium according to the invention is in liquid form. In fact, this form is particularly suitable for the microbiological analysis of food products, which may require a stage of mixing of the solid samples in the culture medium, to permit the release of the microorganisms potentially present in said samples.

Nevertheless, the culture medium can also be in solid form (e.g. an agar-based medium). Identically to the liquid media, the substrates of phospholipase C (PIPLC or PCPLC) can be present in these solid media and permit the detection, identification, or even counting of Staphylococcus aureus.

Alternatively, the culture medium according to the invention can further comprise a substrate that makes it possible to detect enzymatic or metabolic activity of the target microorganisms, different from the phospholipase C activity, such as esterase (notably lipase or phosphatase) coagulase or alpha-glucosidase activity. For direct detection, this substrate can be bound to a moiety serving as a marker, fluorescent or chromogenic. For indirect detection, the culture medium according to the invention can additionally comprise a pH indicator, sensitive to the pH change induced by the consumption of the substrate and revealing the growth of the target microorganisms. Said pH indicator can be a chromophore or a fluorophore.

According to a first embodiment, the selective medium according to the invention is used in microbiological inspection of food products. Preferably, it is used for growing and counting Staphylococcus aureus in milk products.

According to a second embodiment, the selective medium according to the invention is used in microbiological monitoring of the environment. “Environment” means samples of air, samples of water, or samples from surfaces. Among the samples from surfaces, the object of the invention can find particular application in the detection of Staphylococcus aureus in the hospital environment, among the coagulase-positive staphylococci responsible for nosocomial infections.

According to a last embodiment, the selective medium according to the invention is used in clinical analysis for detecting and/or identifying and/or counting Staphylococcus aureus.

Advantageously, the selective medium according to the invention can further comprise a marker of resistance, for example within the scope of a test of the resistance of a strain of Staphylococcus aureus to meticillin.

Another object of the present invention relates to the use of at least one chromogenic, fluorogenic or luminescent substrate of phospholipase C for the differentiation of Staphylococcus aureus bacteria relative to coagulase-negative staphylococci.

Said use is not limited to the manufacture of a culture medium. In fact, it is perfectly conceivable to make identification reagents having one or more substrates of phospholipase C (PIPLC or PCPLC) and permitting the identification of Staphylococcus aureus. Said reagents can be used in products marketed by the applicant such as the VITEK® cards, the API® or RAPiDEC® biochemical test kits.

Another object of the present invention relates to the use of at least one substrate of phospholipase C for preparing a specific culture medium for the growth, detection, identification and/or counting of Staphylococcus aureus bacteria.

Another object of the present invention relates to the use of the culture medium according to the invention, for detecting and/or identifying and/or counting Staphylococcus aureus bacteria in a complex sample.

Finally, a last object of the present invention relates to a method of detection and/or of identification of Staphylococcus aureus bacteria, said method comprising the stages consisting of:
a) Seeding a culture medium according to the invention with a sample that may contain Staphylococcus aureus bacteria;
b) Measuring a change in fluorescence, luminescence or coloration in the culture medium, said change corresponding to the growth of Staphylococcus aureus bacteria in said culture medium.

Advantageously, the method according to the invention comprises an intermediate stage a′) consisting of putting the culture medium thus seeded in conditions suitable for permitting the growth of Staphylococcus aureus bacteria.

According to a particular embodiment, the method according to the invention can comprise a supplementary stage of counting target microorganisms. Said counting stage is preferably performed according to the method of the Most Probable Number (MPN). This method is explained in patent EP 1 105 457 in the name of the applicant.

Preferably, the biological sample is a clinical sample, food sample or environmental sample.

It should finally be noted that application of the objects according to the invention would not be limited to a single type of support. In fact, all the types of supports used in the field of in-vitro diagnostics are suitable for use: microplates, microtubes, microcupules, capillaries, etc.

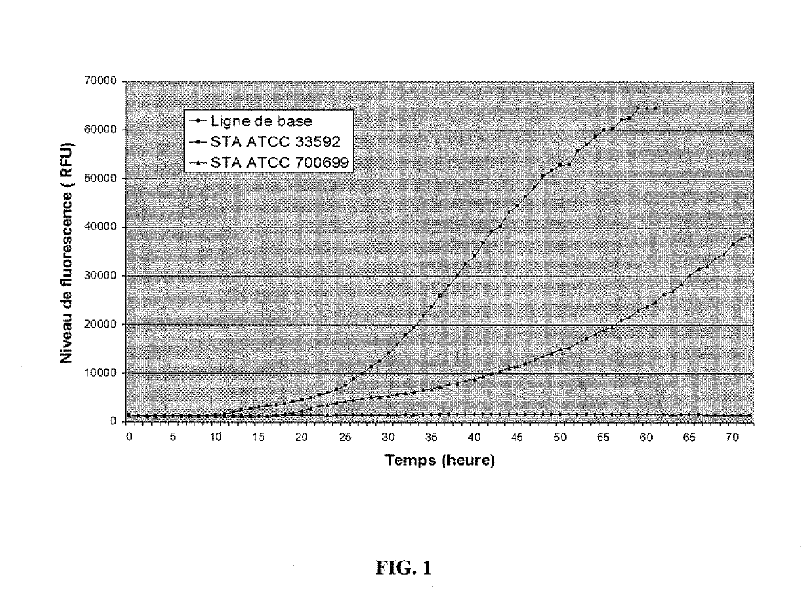

The following examples, in conjunction with FIG. 1, are given for purposes of illustration and are not in any way limiting. They will make it easier to understand the invention.

FIG. 1 shows measurement of fluorescence over time, reflecting the PC-PLC (lecithinase) enzymatic activity of two strains of Staphylococcus aureus

### EXAMPLE 1

### Investigation of the Correlation Between PIPLC Activity and the Presence of a Coagulase in Staphylococcus aureus—Comparison of TEMPO® System/Baird-Parker+RPF (BP+RPF) Medium

43 strains of Staphylococcus aureus and 20 strains of coagulase-negative staphylococci were tested by seeding a Baird-Parker+RPF medium (bioMérieux Ref.: 44003) and a liquid medium derived from the Ottaviani Agosti Agar medium (OAA), used with the TEMPO® system marketed by the applicant.

Medium Derived from OAA:

The medium used is of the following composition:

| Constituent | Concentration in g/l |
|---|---|
| Yeast extract | 5 |
| Biothione | 5 |
| Biosoyase | 5 |
| Biotrypcase | 5 |
| Na pyruvate | 2 |
| Glucose | 0.01 |
| Mg glycerophosphate | 1 |
| NaCl | 5 |
| LiCl | 5 |
| MgCl₂ | 1 |
| MgSO₄ | 0.5 |
| MOPS acid buffer | 14.7 |
| MOPS basic buffer | 7 |
| 4-Methylumbelliferyl myo-inositol-1-phosphate | 0.4 |

MOPS buffer 0.1M at pH 6.70 (adjustment with 6N HCl)
Substrate: 4-Methylumbelliferyl myo-inositol-1-phosphate, N-methyl-morpholine salt, Biosynth, Ref. M-5717 Batch 20078/1)

The culture medium described above contains 4-methylumbelliferyl myo-inositol-1-phosphate, which is combined with a nutrient base to permit, simultaneously with growth, detection of Staphylococcus aureus by the appearance of fluorescence.

### Seeding and Incubation of TEMPO® Cards:

The strains tested, at an initial concentration of 10⁸ Colony Forming Units (CFU)/ml, are diluted in a tryptone salt broth, so as to obtain a final concentration at 10³ CFU/ml. 50 μl of this bacterial suspension is added to 4 ml of culture medium. The whole is loaded in the TEMPO® card, so that the amount of bacterium is 50 CFU/card. The TEMPO® cards are incubated at 37° C. for 24 hours.

### Seeding and Incubation of the Baird-Parker+RPF Medium:

The bacterial suspension at 10³ CFU/ml is also used for seeding the Baird-Parker+RPF medium at a rate of 50 μl.

The medium is incubated at 37° C. for 24 hours.

### Results:

The results obtained with the TEMPO® card are analysed by the TEMPO® system, according to the method of the Most Probable Number (MPN). The appearance of fluorescence is directly linked to the catalytic activity of PIPLC on 4-methylumbelliferyl myo-inositol-1-phosphate leading to release of fluorescent 4-methylumbelliferone.

The colonies obtained on the Baird-Parker+RPF medium are counted conventionally.

Tables 1 and 2 given below summarize the results obtained by the two methods:

TABLE 1
Correlation between PIPLC activity and the presence
of a coagulase in Staphyloccocus aureus and
counts associated with each method (24 h)

| | | | BP + | TEMPO ® | |
|---|---|---|---|---|---|
| | | | RPF count/ | (24 h) | |
| | | Reference | Coagulase | PIPLC | |
| Code | Species | Strain | (24 h) | activity | MPN |
| S1 | S. aureus | 0201058 | 47/+ | + | 30 |
| S2 | S. aureus | 9801012 | 86/+ | + | 112 |
| S3 | S. aureus | 9710057 | 24/+ | + | 44 |
| S4 | S. aureus | 9704027 | 42/+ | + | 40 |
| S5 | S. aureus | 8904051 | 32/+ | + | 28 |
| S6 | S. aureus | 8304011 | 50/+ | + | 40 |
| S7 | S. aureus | 8301044 | 99/+ | + | 112 |
| S8 | S. aureus | 7509008 | 68/+ | + | 56 |
| S9 | S. aureus | 8405022 | 62/+ | + | 72 |
| S10 | S. aureus | 0212024 | 57/+ | + | 56 |
| S11 | S. aureus | 0212017 | 35/+ | + | 44 |
| S12 | S. aureus | 0201059 | 45/+ | + | 21 |
| S13 | S. aureus | 9704026 | 75/+ | + | 128 |
| S14 | S. aureus | 8904052 | 49/+ | + | 12 |
| S15 | S. aureus | 9805029 | 35/+ | + | 19 |
| S16 | S. aureus | 8311065 | 61/+ | + | 39 |
| S17 | S. aureus | 7802083 | 45/+ | + | 26 |
| S18 | S. aureus | 9807062 | 20/+ | + | 28 |
| S19 | S. aureus | ATCC 12600 | 30/+ | + | 21 |
| S20 | S. aureus | CA5 130203 | 41/+ | + | 44 |
| S21 | S. aureus | 201058 | 400/+ | + | 680 |
| S22 | S. aureus | 0201060 | 47/+ | + | 28 |
| S23 | S. aureus | CA20 130203 | 80/+ | + | 92 |
| S24 | S. aureus | CA8 130203 | 49/+ | + | 44 |
| S25 | S. aureus | CA30 130203 | 24/+ | + | 16 |
| S26 | S. aureus | CA40 130203 | 42/+ | + | 44 |
| S27 | S. aureus | CA41 130203 | 75/+ | + | 96 |
| S28 | S. aureus | CA56 130203 | 73/+ | + | 52 |
| S29 | S. aureus | CA9 040303 | 53/+ | + | 56 |
| S30 | S. aureus | CA23 040303 | 85/+ | + | 48 |
| S31 | S. aureus | CA31 040303 | 49/+ | + | 44 |
| S32 | S. aureus | CA32 040303 | 51/+ | + | 84 |
| S33 | S. aureus | LCHA 4890 | 53/+ | + | 44 |
| S34 | S. aureus | Clermont | 33/+ | + | 36 |
| S35 | S. aureus | 57.10 | 40/+ | + | 44 |
| S36 | S. aureus | G52.61 | 48/+ | + | 23 |
| S37 | S. aureus | ATCC 6538P | 31/+ | + | 28 |
| S38 | S. aureus | IM 803 | 32/+ | + | 26 |
| S39 | S. aureus | P02-52 | 67/+ | + | 36 |
| S40 | S. aureus | P5678 | 11/+ | + | 14 |
| S41 | S. aureus | SP2 | 56/+ | + | 36 |
| S42 | S. aureus | 5271 | 35/+ | + | 30 |
| S43 | S. aureus | E1115 | 52/+ | + | 44 |

TABLE 2
Correlation between absence of PIPLC activity and
absence of coagulase in Staphyloccocus spp and
counts associated with each method (40 h)

| | | | BP + | TEMPO ® | |
|---|---|---|---|---|---|
| | | | RPF count/ | (40 h) | |
| | | Reference | Coagulase | PIPLC | |
| Code | Species | Strain | (40 h) | activity | MPN |
| S44 | S. hyicus | 83.01.033 | 38/− | − | <1 |
| S45 | S. epidermidis | ATCC 12228 | 28/− | + | 11 |
| 346 | S. epidermidis | 1566 | 26/+ | + | 26 |
| S47 | S. epidermidis | 0303001 | 30/− | − | <1 |
| S48 | S. xylosus | ATCC 700404 | 7/− | − | <1 |
| S49 | S. schleiferi | 322 | 34/− | − | <1 |
| S50 | S. schleiferi | 4103 | 41/− | − | <1 |
| S51 | S. lugdunensis | 228025 | 60/− | − | <1 |
| S52 | S. intermedius | 217005 | 17/− | − | <1 |
| S53 | S. intermedius | 2885 | 62/− | − | <1 |
| 554 | S. warneri | N930256 | 5/− | − | <1 |
| S55 | S. warneri | Huiller | 1/− | − | <1 |
| S56 | S. haemolyticus | 128549 | 9/− | − | <1 |
| S57 | S. haemolyticus | R202 | 14/− | − | <1 |
| S58 | S. saprophyticus | 42.92 | 20/− | − | <1 |
| S59 | S. saprophyticus | DiMarcheleo | 16/− | − | <1 |
| S60 | S. capriae | 0512901 | 75/− | − | <1 |
| S61 | S. epidermidis | 07.08.901 | 160/− | − | <1 |
| S62 | S. arlettae | 92.03.376 | 159/− | − | 3 |
| S63 | S. lentus | 85.05.027 | 1/− | − | <1 |

All the strains of Staphylococcus aureus tested developed on the Baird-Parker+RPF medium after 24 h of incubation. They all possess a coagulase.
Among the staphylococcus non aureus, only strain S46 shows the presence of a coagulase. Tests performed with an API kit on this strain demonstrated that it is in fact a Staphylococcus aureus.
The S45 strain has a coagulase phenotype—on Baird-Parker+RPF, but a positive signal is observed with TEMPO. Isolation from the dish showed contamination of the sample with Bacillus.
At 24 h, two strains of Staphylococcus aureus (S1 and S15) show a slight variation between the positive signal and the background noise. This is in agreement with the small diameter of the lightening halo around these colonies on Baird-Parker+RPF. Prolonged incubation (40 hours) confirmed the signal to noise ratio is correct.
The PIPLC activity is detected for each of the strains that are positive for coagulase on Baird-Parker+RPF. The coagulase-negative strains do not induce a positive signal for PIPLC activity, except S45.

The results thus obtained therefore clearly demonstrate a correlation between the coagulase activity of Staphylococcus aureus and PIPLC activity.

### EXAMPLE 2

### Analysis of PIPLC Activity in Staphylococcus aureus in Microplates with 2 Different PIPLC Substrates

19 strains of Staphylococcus aureus ATCC are tested for their PIPLC activity in microplates. The tests are performed with two different fluorogenic substrates of PIPLC: 4-methylumbelliferyl-myo-inositol-1-phosphate and 3-chloro-7-hydroxy-4-methylcoumarin myo-inositol-phosphate, added at different concentrations to the constituents of the medium derived from the OAA medium described in example 1.

| Constituent | Concentration in g/l | |
|---|---|---|
| Yeast extract | 5 | 5 |
| Biothione | 5 | 5 |
| Biosoyase | 5 | 5 |
| Biotrypcase | 5 | 5 |
| Na pyruvate | 2 | 2 |
| Glucose | 0.01 | 0.01 |
| Mg glycerophosphate | 1 | 1 |
| NaCl | 5 | 5 |
| LiCl | 5 | 5 |
| MgCl₂ | 1 | 1 |
| MgSO₄ | 0.5 | 0.5 |
| MOPS acid buffer | 14.7 | 14.7 |
| MOPS basic buffer | 7 | 7 |
| 4-Methylumbelliferyl myo-inositol-1- | 0.4 | |
| phosphate | | |
| 3-Chloro-7-hydroxy-4-methylcoumarin myo- | | 0.2 |
| inositol-1-phosphate | | |

4-Methylumbelliferyl myo-inositol-1-phosphate, N-methyl-morpholine salt, Biosynth (Ref. M-5717 Batch 20078/1)
3-Chloro-7-hydroxy-4-methylcoumarin myo-inositol-1-phosphate (PM 458.36)

Synthesis of 3-chloro-7-hydroxy-4-methylcoumarin myo-inositol-1-phosphate

The protected myo-inositol intermediate is first synthesized according to A. V. Rukavishnikov et al., Chem. Phys. Lipids, 89 (1997), 153-157.

This intermediate was then coupled with the second intermediate prepared, namely 3-chloro-7-hydroxy-4-methyl-coumarin-diisopropylphosphoroamidite. The desired product was obtained by a reaction of demethylation of the coupling product in the presence of lithium iodide, then deprotection of the inositol moiety (T. O. Zaikova, et al., Bioconjugate Chem., 12 (2001), 307-313).

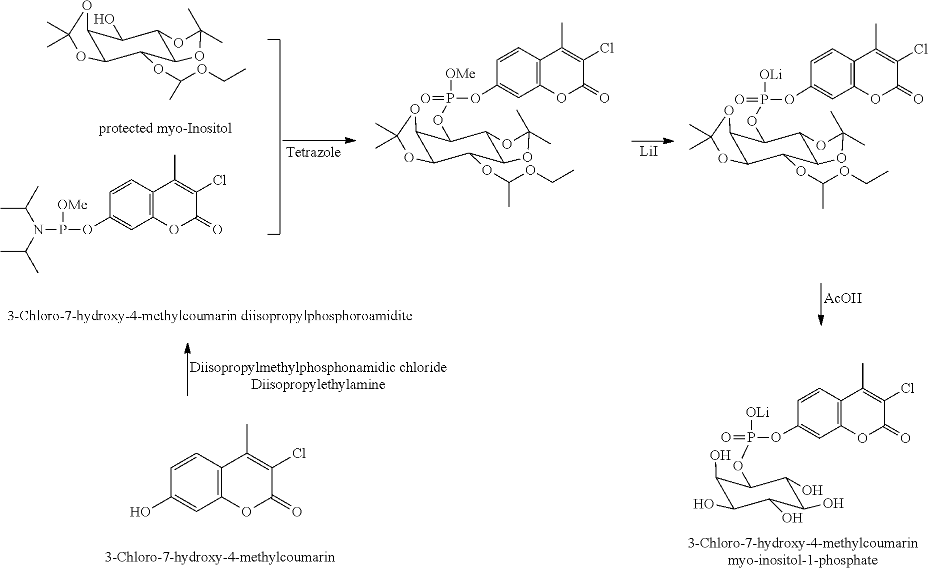

The molecular structures of the final product and of the intermediates were analysed by NMR.

### Seeding and Incubation of the Microplates:

The strains tested, at an initial concentration of 10⁸ (CFU)/ml, are diluted in a tryptone salt broth, so as to obtain a final concentration at 10³ CFU/ml.

10 μl of this bacterial suspension is added to 200 μl of culture medium. Each strain was incubated for 24 hours at 37° C. with each of the media in the microplate reader marketed by the company TECAN under the name GENios™.

### Verification of the Inocula:

In order to verify the inocula, 10 ml of each bacterial suspension at 10³ CFU/ml is used for seeding a Baird-Parker+RPF medium (bioMérieux Ref.: 44003).

### Results:

The results obtained in microplates are summarized in Table 3 below. The + signs correspond to the strains permitting the production of fluorescence at least twice that of the background noise, The − signs correspond to a signal emitted by the medium that is less than the background noise.

TABLE 3

| | PIPLC activity | |
|---|---|---|
| | | 3-Chloro-7-hydroxy-4- |
| | 4-Methylumbelliferyl-myo- | methylcoumarin myo- |
| Strains | inositol-1-phosphate | inositol-1-phosphate |
| ATCC 9144 | + | + |
| ATCC 29213 | + | + |
| ATCC 12598 | + | + |
| ATCC 6538 | + | + |
| ATCC 43300 | + | + |
| ATCC 13150 | + | + |
| ATCC 49775 | − | − |
| ATCC 33862 | + | + |
| ATCC 51740 | + | + |
| CCM 6188 | − | + |
| ATCC 43866 | + | + |
| ATCC 33592 | + | + |
| ATCC 700699 | + | + |
| ATCC 700698 | + | + |
| ATCC 700789 | + | + |
| ATCC 700788 | + | + |
| ATCC 33591 | + | + |
| ATCC 51153 | + | + |
| ATCC 33593 | + | + |

Among the nineteen strains of Staphylococcus aureus ATCC tested, just one does not give any PIPLC signal (ATCC 49775), whatever substrate is used. 17 display PIPLC activity in the presence of 4-methylumbelliferyl-myo-inositol-1-phosphate and 18 display PIPLC activity in the presence of 3-chloro-7-hydroxy-4-methylcoumarin myo-inositol-1-phosphate.

PIPLC activity in Staphylococcus aureus is thus properly confirmed.

It should be noted, moreover, that measurements of PIPLC activity were performed on a total of 109 strains of Staphylococcus aureus (revealed to be coagulase-positive on Baird-Parker+RPF medium) and 26 strains of coagulase-negative staphylococci. The results obtained are summarized in Table 4 below:

TABLE 4

| | | | | Percentage |
|---|---|---|---|---|
| | Number of | Presence of | Absence of | of strains |
| Types of | strains | PIPLC | PIPLC | showing PIPLC |
| strains | tested | activity | activity | activity |
| Staphylococcus | 109 | 105 | 5 | 95.4 |
| aureus | | | | |
| Coagulase- | 26 | 0 | 26 | 0 |
| negative | | | | |
| staphylococci | | | | |

The results presented above confirm that measurement of PIPLC activity is a very relevant parameter for distinguishing Staphylococcus aureus from coagulase-negative staphylococci.

### EXAMPLE 3

### Analysis of PC-PLC (Lecithinase) Activity of Staphylococcus aureus in Microplates (FIG. 1)

The dynamics of the PC-PLC activity of two strains of Staphylococcus aureus ATCC 33592 and ATCC 700699 was evaluated in microplates in the presence of the medium given below. Reading of the appearance of fluorescence in each of the various wells of the microplate was then carried out in kinetic conditions.

1. MEDIA

The medium used is of the following composition, pH 7.2:

| Compounds | Concentration in g/l |
|---|---|
| Yeast extract | 5 |
| Potato peptone | 2 |
| Rhodorsil | 0.4 |
| Manganese sulphate | 1 |
| Sodium pyruvate | 2 |
| Magnesium glycerophosphate | 1 |
| Histidine | 3 |
| NaCl | 5 |
| HEPES basic buffer | 13.8 |
| HEPES acid buffer | 11.92 |
| 4-Methylumbelliferyl choline-phosphate (4MU-CP) | 0.4 |
| Colistin | 0.01 |

4MU-CP is produced by the company Biosynth, under reference M-5528.

2. ASSAY

10 CFU of Staphylococcus aureus ATCC 33592 and ATCC 700699 were inoculated in the wells of the microplate in the presence of the medium described above. The microplate was then incubated at 37° C. in a TECAN reader to evaluate the PC-PLC activity of the two strains of Staphylococcus aureus in the form of the kinetics of hydrolysis of the 4MU-CP substrate, i.e. appearance of fluorescence.

3. RESULTS & INTERPRETATION

Measurement of the appearance of fluorescence following hydrolysis of the 4MU-CP substrate was carried out over an incubation time of 72h for the two strains of Staphylococcus aureus tested (STA ATCC 33592 and STA ATCC 700699) versus the uninoculated control medium (baseline).

The dynamics of PC-PLC activity of the two strains of Staphylococcus aureus investigated is represented on the graph shown in FIG. 1.

Thus, the use of the fluorogenic substrate of PC-PLC, 4MU-CP, permits the detection and discrimination of Staphylococcus aureus.

1. Specific culture medium for growth, detection, identification and/or counting of Staphylococcus aureus bacteria, said medium being comprising: at least one fluorogenic, chromogenic or luminescent substrate of phospholipase C.

2. Culture medium according to claim 1, wherein the substrate of phospholipase C is a substrate of phosphatidylinositol phospholipase C (PIPLC).

3. Culture medium according to claim 2, wherein the concentration of the substrate of PIPLC is between 0.01 and 1 g/l.

4. Culture medium according to claim 2, wherein the substrate(s) of PIPLC are taken from the group comprising: 4-nitrophenyl myo-inositol-1-phosphate, 4-methylumbelliferyl myo-inositol-1-phosphate, 3-chloro-7-hydroxy-4-methylcoumarin myo-inositol-1-phosphate, 3-ethoxycarbonyl-4-methylcoumarin myo-inositol-1-phosphate, 3-cyano-4-methylcoumarin myo-inositol-1-phosphate.

5. Culture medium according to claim 1, wherein the substrate of phospholipase C is a substrate of phosphatidylcholine phospholipase C (PCPLC).

6. Culture medium according to claim 5, wherein the concentration of the substrate of PCPLC is between 0.01 and 1 g/l.

7. Culture medium according to claim 5, wherein the substrate(s) of PCPLC are taken from the group comprising: 5-bromo-4-chloro-3-indoxyl choline phosphate, 3-indoxyl choline phosphate, 4-methylumbelliferyl choline-phosphate.

8. A method for differentiation of Staphylococcus aureus bacteria relative to coagulase-negative staphylococci, comprising:
providing at least one substrate of phospholipase C.

9. (canceled)

10. (canceled)

11. Method of growing, detecting, identifying and/or counting Staphylococcus aureus bacteria, said method comprising the stages consisting of:
seeding a culture medium according to claim 1, with a sample that may contain Staphylococcus aureus bacteria;
measuring a change in the level of fluorescence, luminescence or coloration in the culture medium, said change corresponding to the growth of Staphylococcus aureus bacteria in said culture medium.

12. Method according to claim 11, comprising an intermediate stage a′) consisting of putting the culture medium thus seeded in conditions suitable for permitting the growth of said bacteria.

13. Method according to claim 12, comprising a supplementary stage of counting of Staphylococcus aureus.

14. Method according to claim 11, wherein the biological sample is a clinical sample, food sample or environmental sample.
